(19) United States
(12) Patent Application Publication
(10) Pub. No.: US 2007/0088253 A1
(43) Pub. Date: Apr. 19, 2007

(54) Cannula with extendable distal tip

(76) Inventors: Vahe S. Yacoubian, Glendale, CA (US); Mehmet Bicakci, Santa Ana, CA (US); Michael Webb, Mission Viejo, CA (US)

Correspondence Address: Roger C. Turner, Suite 306, 280 Cagney Ln., Newport Beach, CA, 92663, US

(21) Appl. No.: 11/251,328

(22) Filed: Oct. 14, 2005

(51) Int. Cl. A61M 31/00; A61M 37/00

(52) U.S. Cl. 604/93.01

(57) ABSTRACT

A vent cannula includes a tubular base having a primary longitudinal passage therein with a primary distal tip for insertion into a body vessel. The base has a tubular junction interconnected and extending at an acute angle having a secondary passage therein communicating with the primary passage. A flexible tube is slidably retained within the secondary passage. The flexible tube has a flexible distal portion with a flexible distal tip thereon, a flexible proximal portion with a side port therein and with a proximal handle. The flexible tube is initially positioned at a “first position” wherein the flexible distal tip is within the secondary passage; and is slideable to a “second position” wherein the side port of the flexible proximal portion is at the junction of the primary and secondary passages, and the flexible distal tip is inserted into the primary passage and extended from the primary distal tip to thereby extend the functional tip of the cannula. In a first embodiment, the proximal portion of the flexible tube is sealed within the base by a flexible sleeve; and in a second embodiment, the flexible tube is sealed with elastomeric washers retained between the base and an end cap.

### BACKGROUND OF THE INVENTION

The invention relates to medical cannulas; particularly to medical cannulas used in cardiac surgery.

Cannulas and catheters are commonly used throughout numerous medical and surgical procedures to transfer fluids in and out of vessels and cavities of a patient. In open heart surgery, a cardiopulmonary by pass support system is used to temporarily replace the functions of the heart and lungs by supplying oxygenated blood to the patient's circulatory system. While the patient is on the support system, the ascending aorta is clamped off and the heart can be stopped to facilitate its repair. An aortic cardioplegia cannula is initially inserted into the ascending aorta for supplying anterograde cardioplegia solution to the heart. The heart also receives retrograde cardioplegia from a different source to facilitate perfusion of the heart. The heart is stopped and can then be repaired with one or more anastomoses of a vessel graft (typically a saphenous vein) creating a new blood flow path directly from the ascending aorta to the heart. This procedure is known as “coronary bypass” around the blocked coronary arteries.

This surgical procedure typically utilizes a cardioplegia/vent cannula having a proximal “Y” junction whereby cardioplegia solution can be initially supplied to the heart, then when desired, the cardioplegia line can be closed off, and the vent line (connected to a vacuum system) can be opened to evacuate air and blood from the ascending aorta to facilitate the anastomoses surgical procedures. A device typical of such prior art cardioplegia/vent cannulas is described in U.S. Pat. No. 4,596,552 entitled Cardioplegia Cannula by DeVries. This cannula in the patent to DeVries illustrates a typical distal tip, having a fixed length, which extend a relatively short distance into the aorta.

Recent data shows that minimizing the manipulation of the ascending aorta during surgery minimizes the chances of embolization and possible “stroke”. Most surgeons therefore typically only apply the cross clamp once, and perform all proximal and distal anastomoses during the same cross clamp application. When this technique is used, the patient continues to receive retrograde cardioplegia while the proximal anastomoses are being performed. The patient and the ascending aorta are naturally in the supine, horizontal orientation; thus, the aortic root fills up with blood coming from the left main and right main coronary arteries. The operating field is usually flooded with blood, impairing adequate visibility of the field by the surgeon. The aortic cannula is then vented to keep the field as dry and clear as possible. The present aortic cannulas have a fixed length intra-aorta distal tip, which extend a fixed (relatively short) distance into the aorta. The short length is required to allow the cannula to be inserted into the vessel by a trocar (large tubular needle), extended through the distal tip of the cannula, without damage to the opposed wall of the vessel.

The present cannulas typically are able to clear the blood only from the upper region of the aorta. This limited venting may not provide optimum visibility for the proximal anastomoses procedures, and may preclude possible advantageous proximal anastomoses along the mid and lower sides of the aorta. It would be highly desirable to have a vent cannula with a distal tip that could be extended, if necessary, to more effectively clear the surgical field of the aorta during heart surgery.

In view of the foregoing, it is an object of the present invention, to provide a cannula having an extendable distal tip.

It is another object to provide an aortic vent cannula having an extendable distal tip, and which is compatible with current introducer devices, cardioplegia flow and suction features of current cardioplegia cannulas.

### SUMMARY OF THE INVENTION

The foregoing objects are accomplished by the present invention of an improved vent cannula having an extendable tip for venting and clearing a surgical field. Briefly, the vent cannula comprises a tubular base having a primary passage therein with a distal end and having an interconnected, angled secondary passage therein, and a flexible tube slidably positioned within the secondary passage than is extendable into the primary passage and through the distal end to extend the functional tip of the cannula.

In more detail, the vent cannula includes a tubular base having a primary longitudinal passage therein with a primary distal tip for insertion into a body vessel. The base has a lateral flange for supporting and securing the base to the exterior wall of the vessel, and has a proximal primary tubular portion having a primary proximal end for further connection to a fluid transfer system.

The base further has a tubular junction extending at an acute angle from the proximal primary portion and having a secondary passage therein communicating with the primary passage. The tubular junction further has a proximal secondary portion with a secondary proximal end.

A flexible tube is slidably retained within the secondary passage. The flexible tube has a flexible distal portion with a flexible distal tip thereon, a flexible proximal portion with a side port therein and with a proximal handle. The flexible tube is initially positioned at a “first position” wherein the flexible distal tip is within the secondary passage; and is slideable to a “second position” wherein the side port of the flexible proximal portion is at the junction of the primary and secondary passages, and the flexible distal tip is inserted into the primary passage and extended from the primary distal tip to thereby extend the functional tip of the cannula.

The cannula further comprises means for sealing the flexible tube within the secondary proximal end of the base. Such sealing means are provided in examples described as a first embodiment and a second embodiment of the present invention

A first embodiment of the cannula includes the forgoing base and flexible tube assembly and further includes a sealing means comprising a flexible collapsible sleeve. The sleeve has a proximal end connected and sealed onto the flexible proximal handle of the flexible tube, and has a distal end connected and sealed onto the secondary proximal end of the base. The sleeve provides a positive air tight seal of the cannula at all times, and particularly during any repositioning of the flexible tube within the base.

A second embodiment of the cannula includes the forgoing base and flexible tube assembly and further includes a sealing means wherein the flexible proximal portion of the flexible tube is slidably sealed within sealing washers retained between the secondary passage and an end cap.

In more detail, in the second embodiment the base further includes a secondary tubular member with an annular end flange thereon; and a cylindrical cap having an inner diameter engagable with the end flange and having a central aperture therein with a diameter corresponding to the diameter of the flexible proximal portion of the flexible tube. One or more elastomeric sealing washers are interposed between the end flange and the end cap. The flexible tube is thereby sealed and slideable within the secondary proximal end of the base.

The cannulas of the present invention are fully compatible with other standard components (used with conventional vent cannulas). The primary tubular proximal end of the base further includes an end fitting thereon for initially attaching a trocar within the primary passage for inserting the primary distal tip of the base into the vessel. After insertion, the trocar is removed and the end fitting is further used for attaching a standard “Y” junction thereto for use during surgical procedures. The “Y” junction has a first tubular branch for transferring fluids into the vessel and a second tubular branch for venting fluids from the vessel wherein the first branch and the second branch include snap-release shut-off valves for selectively controlling the respective branches of the junction.

### BRIEF DESCRIPTION OF THE DRAWINGS

While the novel features of the invention are set forth in the appended claims, the invention will be better understood along with other features thereof from the following detailed description taken in conjunction with the drawings, in which:

FIG. 1 is a front perspective view of a first embodiment of the cannula of the present invention;

FIG. 2 is an exploded, partially sectional view of FIG. 1;

FIG. 3 is a front elevational sectional view of the invention of FIG. 1, illustrating the distal tip of the cannula in a first position;

FIG. 4 is similar to FIG. 3, showing the distal tip of the cannula in an extended, second position;

FIG. 5 is a front perspective view, similar to FIG. 1, in cross section, also showing the distal tip of the cannula in the first position;

FIG. 6 is similar to FIG. 5, also showing the distal tip of the cannula in the extended, second position;

FIG. 7 is a front perspective view of a trocar, for use with the present invention;

FIG. 8 is a front perspective view of the first embodiment including the trocar of FIG. 7, inserted into the cannula, for introducing the distal tip of the cannula into a vessel (with a “Y” junction shown positioned to be substituted for the trocar);

FIG. 9 is a front perspective view of the first embodiment of the present invention with the “Y” junction connected thereto for reperfusion and for venting fluids during surgery;

FIG. 10 is a front perspective view of second embodiment of the cannula of the present invention;

FIG. 11 is an exploded perspective view of the flexible tube and sealing means for the flexible tube shown in FIG. 10;

FIG. 12 is a front perspective view, similar to FIG. 10, partially in cross section, showing the distal tip of the cannula in a first position; and

FIG. 13 is similar to FIG. 12, showing the distal tip of the cannula in an extended, second position.

### DETAILED DESCRIPTION OF THE INVENTION

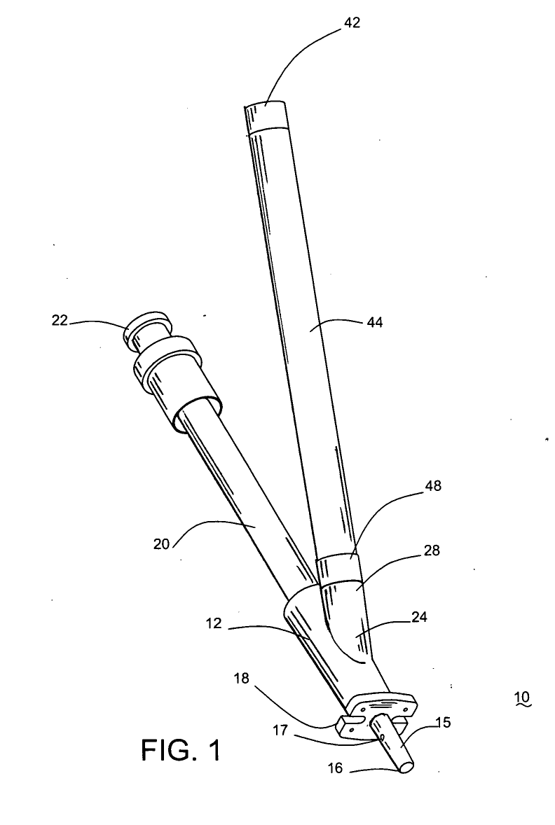

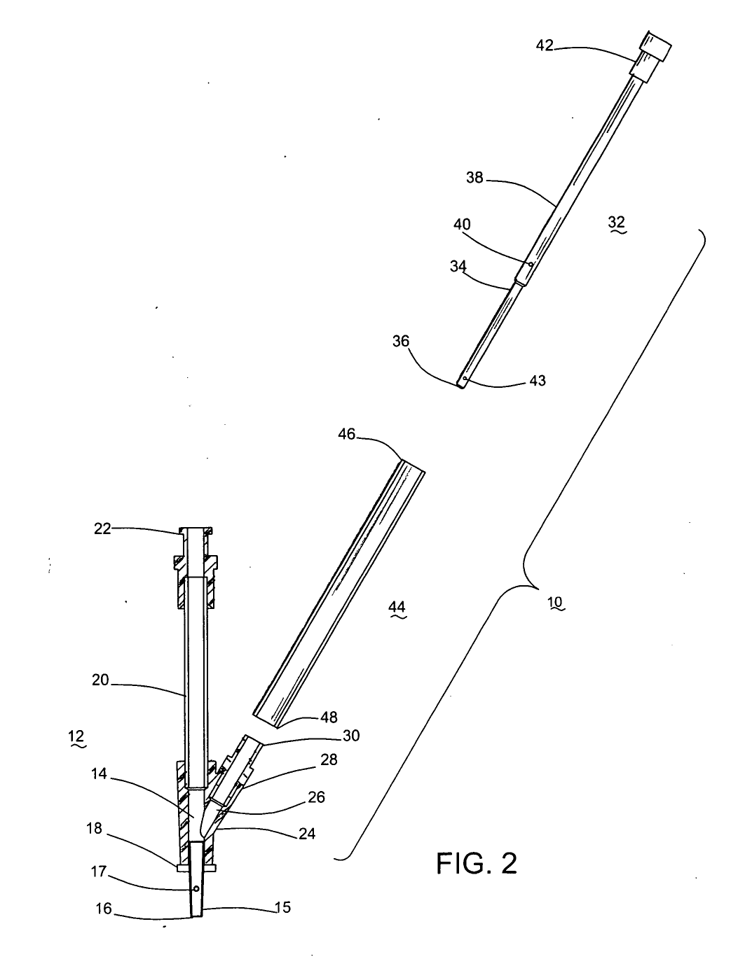

Referring first to FIGS. 1 and 2, there is illustrated a cannula 10 as an example of a first embodiment of the present invention The cannula 10 can have broad applications for medical cannulas and catheters, and for non-medical applications for tubes having a primary passageway with a fixed distal tip, wherein the distal tip is advantageously extendable and functional through the primary passageway. The current best use of the invention is as a vent cannula for clearing a surgical field during cardiac surgery. The invention is known as the Yacoubian “OPTICLEAR” Vent Cannula, developed and currently marketed by CalMed Laboratories, Inc., of Costa Mesa, Calif. This Detailed Description of the Invention is an example of such a medical vent cannula

The cannula 10 comprises, a tubular base 12 having a primary longitudinal passage 14 therein. The base has a primary distal portion 15, with a primary distal tip 16 thereon with aspiration vents 17 therein, and having a lateral flange 18. The base includes a proximal primary tubular portion 20 having a proximal primary end 22 for further connection to a fluid transfer system. The primary distal tip extends about 0.50 inches from the lateral flange, for inserting through the upper wall of the vessel (and through any layer of atherosclerotic plaque that may be present on the inner wall), and into the interior of the vessel. The lateral flange is used for supporting and securing (suturing) the base to the exterior wall of the vessel.

The base 12 further has a tubular junction 24 extending at an acute angle from the primary passage 14 and having a secondary passage 26 therein communicating with the primary passage. The junction further has a proximal secondary portion 28 having a proximal secondary end 30. The junction extends preferably at an acute angle of about 30 degrees; however, the angle can range from about 10 to 50 degrees, or can be arcuate and tangential, as desired for a smooth communication path of the secondary passage with the primary passage.

The cannula 10 further comprises an extendable flexible tube 32 slidably retained within the secondary passage 26. The flexible tube has a flexible distal portion 34 having a diameter corresponding to the inner diameter of the primary distal portion 15, with a flexible distal tip 36; and has a flexible proximal portion 38 having a diameter corresponding to the diameter of the primary passage 14. The flexible proximal portion has several (about two to four) radial side ports 40 therein, and has a flexible proximal handle 42. The flexible distal tip is preferably rounded to facilitate maneuvering within the passages of the cannula and within the vessel, and preferably further includes radial apertures 43 to facilitate venting. The flexible distal portion has a length suitable for extending the flexible distal tip from the junction of the first passage 14 and secondary passage 26, through the lower portion of the primary passage 14 and primary distal portion 15, to an extendable length of about 0.40 inches beyond the primary distal tip 16. In the current example, the flexible distal portion is a total of about 1.50 inches. The flexible proximal portion has a length that extends from the proximal secondary end 30 of the base, at least the length of the flexible distal portion (about 1.50 inches as discussed above) and can be longer to provide easy manipulation of the flexible proximal handle 42. A suitable overall length of the flexible tube is about 4.00 inches.

The slidable engagement of the flexible tube 32 within the passages of the base 12 could possibly be precisely produced whereby a seal is maintained within the base during manipulation of the flexible tube; however it is preferable to provide a means for sealing the flexible tube within the secondary proximal end of the base. A suitable sealing means is provided by a flexible collapsible sleeve 44 surrounding the flexible proximal portion of the flexible tube and enclosing it within the base. The sleeve has a proximal end 46 connected and sealed onto the proximal handle 42 of the flexible tube, and has a distal end 48 connected and sealed onto the proximal secondary end 30 of the base.

The sleeve 44 can be generally cylindrical as shown or can be formed generally cylindrical and having alternating reduced diameters that are uniformly spaced apart (somewhat as shown in FIG. 4), and having distal and proximal ends connected and sealed as described above. Such alternating reduced diameters result in annular accordion-type folds that are uniformly expandable and contractible with movement of the flexible tube, to seal the flexible tube within the proximal secondary end of the base.

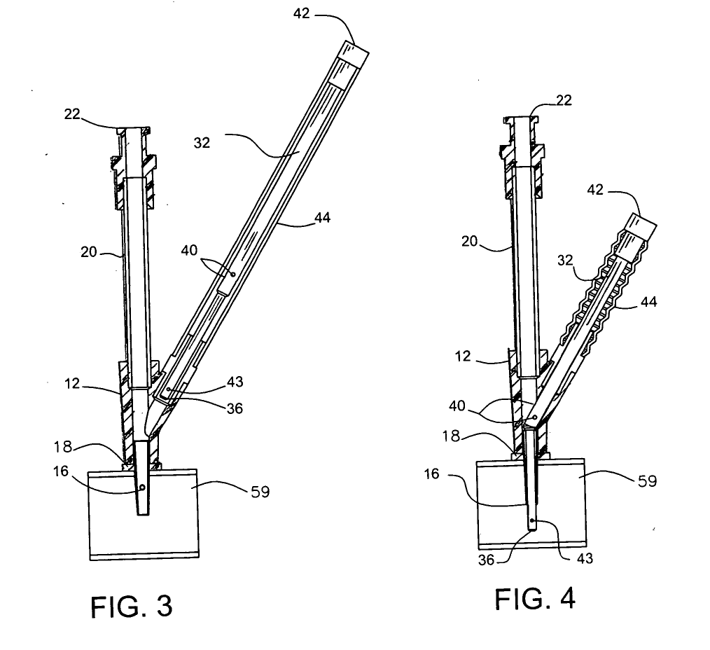

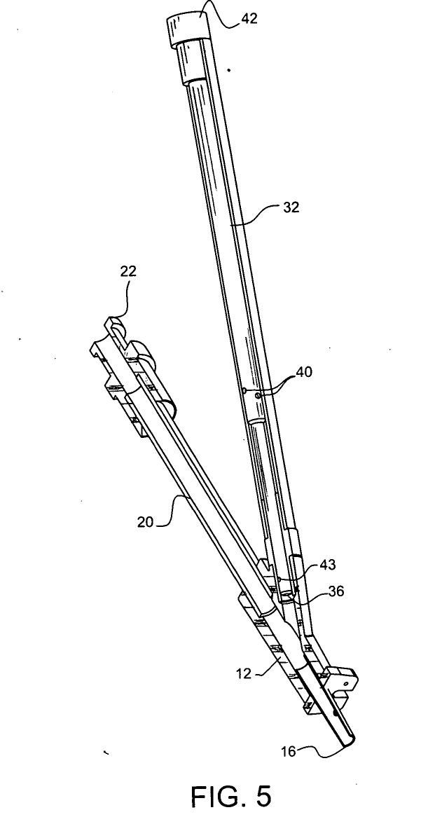

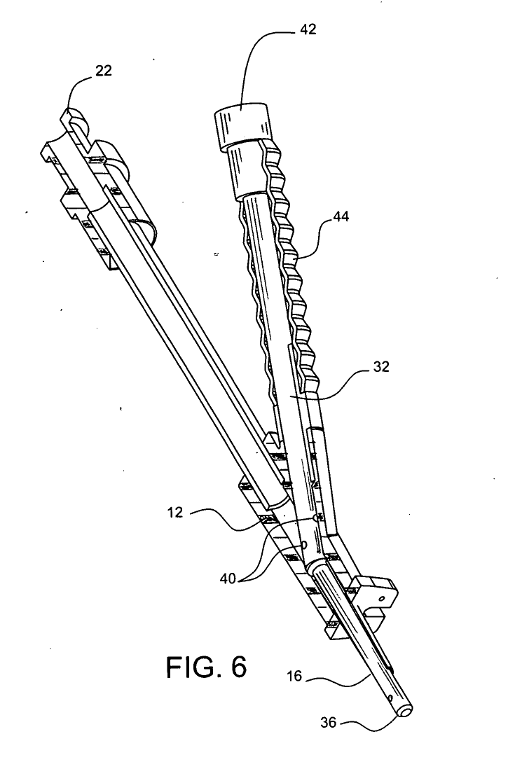

Referring also to FIGS. 3-6, the flexible tube 32 is initially oriented at a first position (see FIGS. 3 and 5) wherein the flexible distal tip 36 is within the secondary passage 26; and is slidable to a second position (see FIGS. 4 and 6) wherein the flexible tube is advanced into the primary passage 14, and the flexible distal tip is extended through the primary distal tip 16 of the base 12, to thereby extend the functional length of the distal tip of the cannula: And further, wherein the side ports 40 of the flexible proximal portion 38 are positioned at the junction of the primary and secondary passages of the base, whereby the flexible distal tip is in communication with the primary passage. A vacuum applied to the primary proximal end 22 will vent any fluids surrounding the extended flexible distal tip. The side ports 40 are preferably offset and arranged radially to insure that at least one of the side ports is open to the primary passage. The radial side ports can be further arranged along the length of the flexible distal portion to provide communication with the primary passage at variable lengths of extendability of the cannula 10.

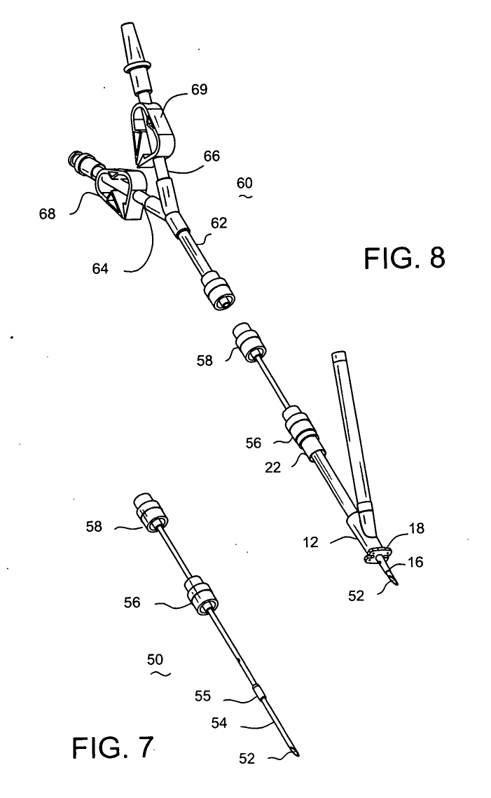

Referring also to FIGS. 7-9, the operation of the improved cannula 10 is discussed in more detail. The cannula is typically introduced into the vessel with a tubular trocar 50 (see FIG. 7). The trocar has a angled sharp distal point 52, a distal portion 54 with a shoulder 55 thereon; and having an intermediate lure lock type fitting 56, and a proximal lure lock type fitting 58 at the proximal end of the trocar. As shown in FIG. 8, the trocar is inserted into the primary passage 14 of the cannula and is secured with the intermediate fitting 56 to a mating fitting at the proximal primary end 22 of the cannula. The length of the distal portion (the shoulder 55 and intermediate fitting 56) is precisely fixed so that the sharp distal point extends just beyond the primary distal tip 16 of the cannula.

Referring particularly to FIG. 3, the cannula and trocar assembly is then positioned at the desired location on the upper outer wall of the ascending aorta 59, and the trocar is pushed into the wall of the aorta making a small incision (not shown), and introducing the primary distal tip 16 through the wall of the aorta and any inner layer of plaque, and into the interior of the aorta. The length of the primary distal tip is predetermined to be a relatively short length (about 0.50 inches) to insure that inter aortic length of the primary distal tip and the distal point 52 of the trocar does not extend so as to damage the opposed inner wall of the aorta The lateral flange 18 is then secured to the outer wall with several “purse string” type sutures, forming a hemostatic seal around the cannula The proximal fitting 58 of the trocar can be further attached to a system for delivering or removing fluids from the aorta; however, the trocar is usually then removed and discarded, and replaced with a “Y” junction 60 to proceed with the procedure.

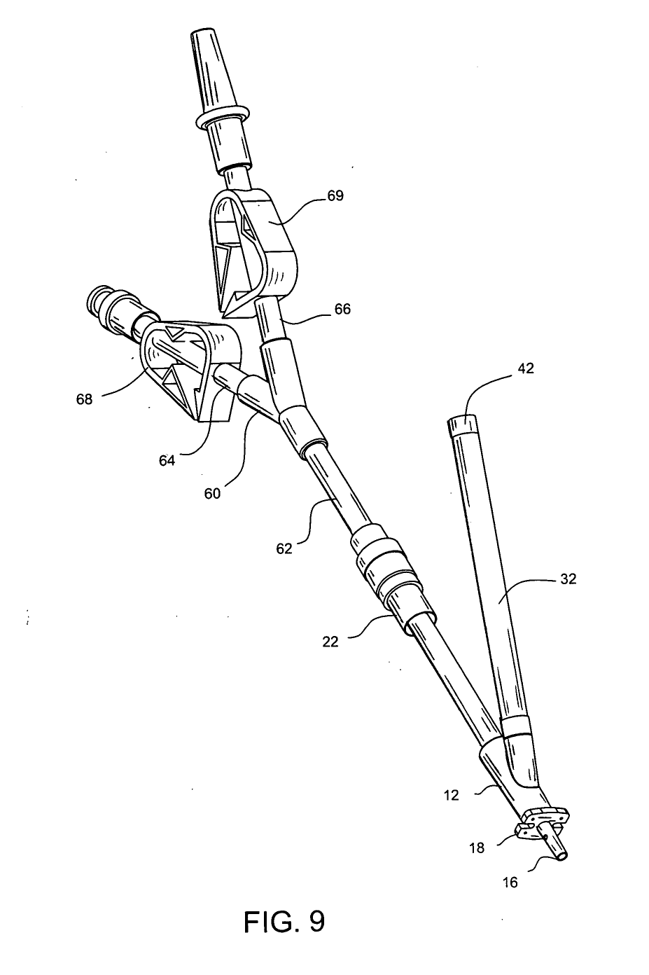

Referring particularly to FIG. 9, the cannula 10 and “Y” junction 60 are shown assembled with distal primary branch 62 connected to the proximal primary end 22 of the cannula. The “Y” junction further includes a (left) proximal perfusion branch 64 with suitable fitting for attachment to a perfusion line for transferring fluids into the aorta; and a (right) proximal vent branch with suitable fitting for attachment to a suction line for removing fluids for venting and clearing the aorta. The proximal branches further include snap-release shut-off valves, typically a blue valve 68 on the perfusion branch, and typically a red valve 69 on the vent branch, for respectively controlling the fluids into and out of the cannula, and thus the aorta.

During a typical surgical procedure, the vent branch 66 is initially snapped closed, and cardioplegia solution is initiated through the perfusion branch 64 that is snapped open. After sufficient perfusion has taken place, the perfusion branch is snapped closed. The surgeon then prepares to perform a proximal anastomosis of a venous graft onto the aorta. As previously discussed, when this technique is used, the patient has been receiving retrograde cardioplegia, and the aortic root fills up with blood coming from the left main and right main coronary arteries. The operating field is usually flooded with blood, impairing adequate visibility of the field by the surgeon. The vent branch of the cannula is then opened and activated to vent and clear the operating field.

The fixed length of the primary distal tip 16 of the cannula extends a fixed (relatively short) distance into the aorta, and does not always adequately vent the lower portion of the surgical field (see FIGS. 3 and 5). The flexible tube 32 of the present invention is then advanced by pressing the proximal handle 42. The flexible distal tip 38 slides into the primary passage and through the primary distal tip to the second position (an additional 0.40 inches) into the aorta to more effectively clear the surgical field (see FIGS. 4 and 6).

The proximal anastomoses can then be efficiently and safely performed onto the wall of the ascending aorta. Upon completion of the anastomoses, the proximal handle 42 is pulled outward, and the flexible tube 32 is thereby retracted to the first position so that the air can be vented from the upper portion of the aorta through the aspiration vents 17 in the primary distal tip 16 of the cannula. The cannula is then removed and the insertion opening in the aorta is sealed with suitable sutures.

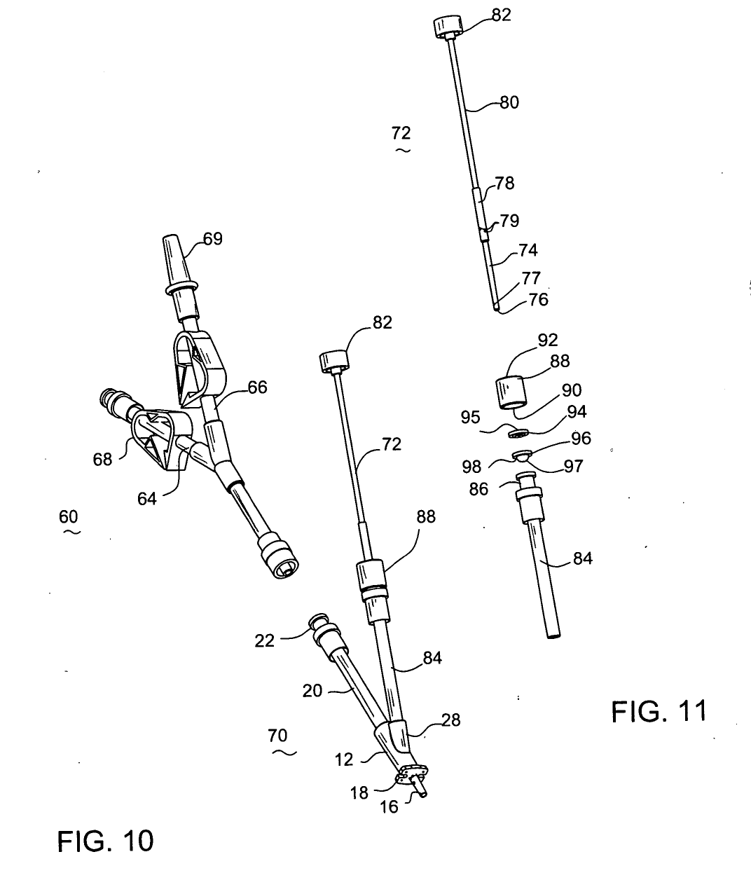

Referring now to FIGS. 10-12, there is illustrated a cannula 70 as an example of a second embodiment of the present invention The second embodiment is similar to the first embodiment except that the means for sealing an extendable flexible tube 72 within the secondary proximal end 30 of the base 12 is more nagged and robust.

The cannula 70 includes essentially the same base 12 having the primary passage 14, the junction 24 having secondary passage 26, the primary proximal tube portion 20, the proximal end fitting 22, and the proximal secondary portion 28, as previously discussed. (However, cannula 70 does not include the secondary end 30 of the base, or the flexible collapsible sleeve 44 as in cannula 10.)

The junction 24 includes the secondary proximal portion 28 and further includes a secondary tubular member 84 extending therefrom for further retaining the flexible tube 72. The tubular member has an annular end flange 86 (forming a suitable fitting) thereon. The cannula 70 further includes a cylindrical end cap 88 having an inner diameter 90 forming a suitable fitting for engaging the end flange 86, and having a central aperture 92 therein with a diameter corresponding to the diameter of the flexible proximal portion of the flexible tube 72.

The cannula 70 utilizes the extendable flexible tube 72 further having a flexible distal portion 74 with a flexible distal tip 76 thereon; a flexible middle portion 78 having several radial side ports 79 therein, and a flexible proximal portion 80 having a flexible proximal handle 82. The flexible distal portion is slideable retained within the secondary passage 26 and the secondary tubular member 84, With the flexible proximal handle 82 extending from the secondary tubular member. The flexible distal tip 76 is preferably rounded to facilitate maneuvering within the passages of the cannula and within the vessel and preferably further includes radial apertures 77 to facilitate venting. The flexible distal portion has a length suitable for extending the flexible distal tip from the junction of the first passage 14 and secondary passage 26, through the lower portion of the primary passage 14 and primary distal portion 15, to an extendable length of about 0.40 inches beyond the primary distal tip 16. In the current example, the flexible distal portion is about 1.25 inches. The flexible intermediate portion extends for about 1.00 inch, and the flexible proximal portion has a length that extends from the proximal secondary end 30, through the proximal tubular member 84 of the base, and at least the length of the flexible distal portion (about 1.25 inches as discussed above) to provide easy manipulation of the flexible proximal handle 82. A suitable overall length of the flexible tube is about 5.00 inches.

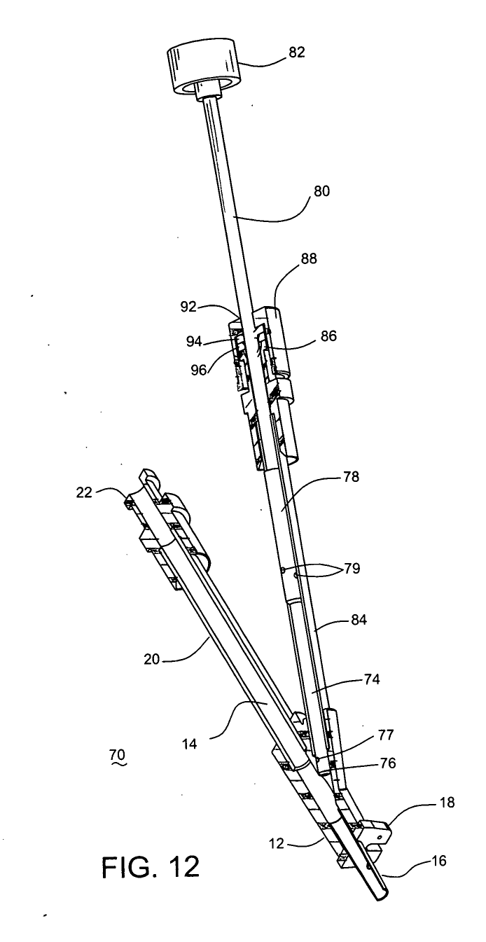

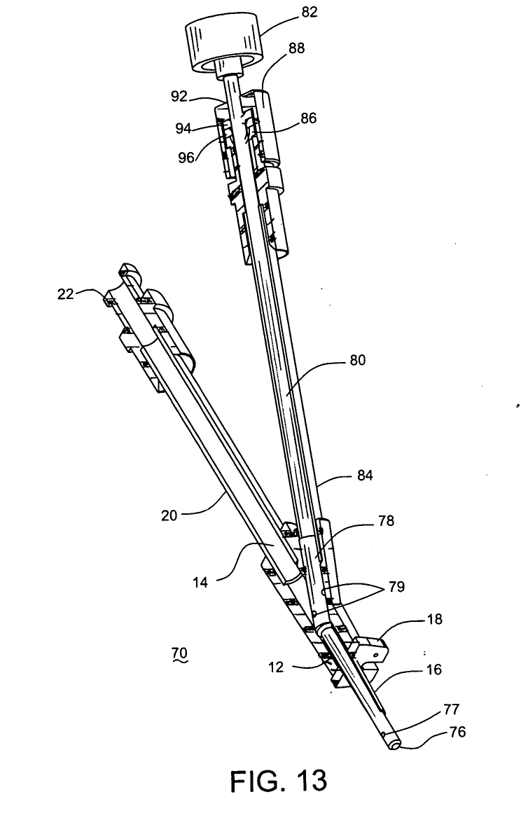

Referring particularly to FIGS. 12 and 13, (and as previously discussed in reference to the first embodiment) the flexible tube is initially oriented in a first position (see FIG. 12) wherein the flexible distal tip 76 is within the secondary passage 26; and is slideable to a second position (see FIG. 13) wherein the flexible tube is advanced into the primary passage 14, and the flexible distal tip 76 is extended through the primary distal tip 16 of the base 12, to thereby extend the functional length of the distal tip of the cannula. And further, wherein the side ports 79 of the flexible middle portion 78 are positioned at the junction of the primary and secondary passages of the base, whereby the flexible distal tip is in communication with the primary passage. A vacuum applied to the primary proximal end 22 will vent any fluids surrounding the extended flexible distal tip. The side ports 79 are preferably offset and arranged radially to insure that at least one of the side ports is open to the primary passage. The side ports can be further arranged along the length of the flexible distal portion to provide variable lengths of extendibility of the cannula 70.

The flexible proximal portion 80 of the extendable flexible tube 72 is sealed within the secondary tubular member 84 by one or more elastomeric sealing washers interposed between the end flange 86 and the end cap 88. For example, as particularly shown in FIG. 11, a first elastomeric sealing washer 94 in the form of a disc having an outer diameter corresponding to the inner diameter 90 of the end cap 88, and having a central opening 95 (smaller than the diameter of the flexible proximal portion) is positioned on the flexible proximal portion of the flexible tube.

Another suitable example is a second elastomeric sealing washing 96 having a flexible hemispherical distal diaphragm 97 with a flanged outer diameter 98 corresponding to the inner diameter of the end cap 88 and with an opening therein (preferably radial slits forming a leaflet type valve) for sealing the diameter of the proximal portion of the flexible tube.

A variety of single and multiple elastomeric sealing washers can provide a suitable seal. It has been found that the combination of the first washer 94 positioned on the flexible proximal behind the second washer 96, and both interposed between the end flange 86 and the end cap 88, provides an excellent slideable sealed engagement of the extended flexible tube within the base of the cannula 70.

As discussed in reference to the first embodiment, the cannula 70 further initially utilizes the proximal primary tubular portion 20 with a trocar 50 (see FIG. 7) for inserting the primary distal tip of the base of the cannula into the vessel.

As discussed in reference to the first embodiment, the cannula 70 finally utilizes the proximal primary tubular portion 20 with the “Y” junction 60 (see FIG. 10) having the first tubular branch 64 for transferring fluids into the vessel, and the second tubular branch 66 for venting fluids from the vessel. The first branch and the second branch each includes the snap-release shut-off valve as typical means for selectively clamping off the respective branches of the junction

Thus, first and second embodiments have been described as examples of the present invention wherein a vent cannula includes an extendable distal tip that can be extended, if necessary, to more effectively clear the surgical field during cardiac surgery. The extendable distal tip cannulas of the present invention are completely compatible with the insertion, use and procedures as used with current cardioplegia/vent cannulas.

While specific embodiments and examples of the present invention have been illustrated and described herein, it is realized that modifications and changes will occur to those skilled in the art. It is therefore to be understood that the appended claims are intended to cover all such modifications and changes as fall within the spirit and scope of the invention.

### ELEMENTS AND FEATURES

Elements and features

| | |
|---|---|
| 10 | Cannula (first embodiment) |
| 12 | tubular base |
| 14 | primary passage |
| 15 | primary distal portion |
| 16 | primary distal tip |
| 17 | aspiration vents |
| 18 | lateral flange |
| 20 | proximal primary tubular portion |
| 22 | proximal primary end |
| 24 | tubular junction |
| 26 | seconndary passage |
| 28 | proximal secondary portion |
| 30 | proximal secondary end |
| 32 | flexible tube |
| 34 | flexible distal portion |
| 36 | flexible distal tip |
| 38 | flexible proximal portion |
| 40 | side ports |
| 42 | flexible proximal handle |
| 43 | radial apertures |
| 44 | flexible collapsible sleeve |
| 46 | sleeve proximal end |
| 48 | sleeve distal end |
| 50 | trocar |
| 52 | sharp distal point |
| 54 | distal portion |
| 56 | intermediate fitting |
| 58 | proximal fitting |
| 59 | Aorta |
| 60 | “Y” Junction |
| 62 | distal primary branch |
| 64 | (left) distal perfusion branch |
| 66 | (right) distal vent branch |
| 68 | snap-release shut-off valve (in) |
| 69 | snap-release shut-off valve (vent) |
| 70 | Cannula (second embodiment) |
| 72 | extendable flexible tube |
| 74 | flexible distal portion |
| 76 | flexible distal tip |
| 77 | radial apertures |
| 78 | flexible middle portion |
| 79 | side ports |
| 80 | flexible proximal portion |
| 82 | flexible proximal handle |
| 84 | secondary tubular member |
| 86 | annular end flange (fitting) |
| 88 | cylindrical end cap |
| 90 | inner diameter(fitting) |
| 92 | aperture |
| 94 | first elastomeric sealing washer |
| 95 | central opening |
| 96 | second elastomeric sealing washer |
| 97 | distal hemispherical diaphragm |
| 98 | outer flange |

1. A cannula having an extendable tip, comprising:
a tubular base having a primary longitudinal passage therein with a primary distal tip for insertion into an opening, a lateral flange for supporting and securing the base to the opening, and with a proximal primary tubular portion having a proximal primary end for further connection to a fluid transfer system;
said base further having a tubular junction extending at an acute angle from the proximal primary portion and having a secondary passage therein communicating with the primary passage and having a proximal secondary portion with a proximal secondary end;
a flexible tube slidably retained within the secondary passage having a flexible distal portion with a flexible distal tip thereon, and a flexible proximal portion with a side port therein and with a proximal handle thereon; whereas said flexible tube is slideable from a first position wherein the flexible distal tip is within the secondary passage, to a second position wherein the side port of the flexible proximal portion is at the junction of the primary and secondary passages, and the flexible distal tip is extended from the primary distal tip of said base to thereby extend the functional tip of said cannula.

2. The cannula as defined in claim 1 further comprising means for sealing the flexible tube within the secondary proximal end of said base.

3. The cannula as defined in claim 2 wherein said sealing means comprises a flexible collapsible sleeve having a proximal end connected and sealed onto the proximal handle of said flexible tube, and having a distal end connected and sealed onto the proximal secondary end of said base.

4. The cannula as defined in claim 2 wherein said sealing means further comprises the secondary proximal end of said base having a secondary tubular member with an annular end flange thereon,
a cylindrical cap having an inner diameter engagable with the end flange and having a central aperture therein with a diameter corresponding to the diameter of the flexible proximal portion of said flexible tube, and
at least one elastomeric sealing washer having an outer diameter corresponding to the inner diameter of said cap and having a central opening for sealing the diameter of the proximal portion of said flexible tube, and having said sealing washer interposed between the end flange and said end cap, whereby the flexible tube is sealed and slideable within the secondary proximal end of said base.

5. The cannula as defined in claim 2 wherein said sealing means further comprises the secondary proximal end of said base having a secondary tubular member with an annular end flange thereon,
a cylindrical cap having an inner diameter engagable with the end flange and having a central aperture therein with a diameter corresponding to the diameter of the flexible proximal portion of said flexible tube, and
a first elastomeric sealing washer having an outer diameter corresponding to the inner diameter of said cap and having a central opening corresponding to the diameter of the flexible proximal portion of said flexible tube,
a second elastomeric sealing washer having a flexible hemispherical distal diaphragm with a flanged outer diameter corresponding to the inner diameter of said cap and with an opening therein for sealing the diameter of the proximal portion of said flexible tube; with
said first and second sealing washers interposed between the end flange and said end cap, whereby the flexible tube is sealed and slideable within the secondary proximal end of said base.

6. A vent cannula having an extendable tip for clearing a surgical field, comprising:
a tubular base having a primary longitudinal passage therein with a primary distal tip for insertion into a body vessel, a lateral flange for supporting and securing the base to the exterior wall of the vessel, and with a proximal primary tubular portion having a primary proximal end for further connection to a fluid transfer system;
said base further having a tubular junction extending at an acute angle from the proximal primary portion and having a secondary passage therein communicating with the primary passage and having a proximal secondary portion with a secondary proximal end;
a flexible tube slidably retained within the secondary passage having a flexible distal portion and with a flexible distal tip thereon, and a flexible proximal portion with a side port therein and with a proximal handle thereon; the flexible proximal portion having a length that extends from the secondary proximal end of said base; whereas the flexible tube is slideable from a first position wherein the flexible distal tip is within the secondary passage, to a second position wherein the side port of the flexible proximal portion is at the junction of the primary and secondary passages, and the flexible distal tip is extended from the primary distal tip of said base to thereby extend the functional tip of said cannula; and
means for sealing the flexible tube within the secondary proximal end of said base.

7. The cannula as defined in claim 6, wherein said sealing means comprises a flexible collapsible sleeve having a proximal end connected and sealed onto the flexible proximal handle of said flexible tube, and having a distal end connected and sealed onto the secondary proximal end of said base.

8. The cannula as defined in claim 6, wherein said sealing means further comprises the secondary proximal end of said base having a secondary tubular member with an annular end flange thereon,
a cylindrical cap having an inner diameter engagable with the end flange and having a central aperture therein with a diameter corresponding to the diameter of the flexible proximal portion of said flexible tube, and
at least one elastomeric sealing washer having an outer diameter corresponding to the inner diameter of said cap and having a central opening for sealing the diameter of the proximal portion of said flexible tube, and having said sealing washer interposed between the end flange and said end cap, whereby the flexible tube is sealed and slideable within the secondary proximal end of said base.

9. The cannula as defined in claim 6, wherein said sealing means further comprises the secondary proximal end of said base having a secondary tubular member with an annular end flange thereon;
a cylindrical cap having an inner diameter engagable with the end flange and having a central aperture therein with a diameter corresponding to the diameter of the flexible proximal portion of said flexible tube; and
a first elastomeric sealing washer having an outer diameter corresponding to the inner diameter of said cap and having a central opening corresponding to the diameter of the flexible proximal portion of said flexible tube,
a second elastomeric sealing washer having a flexible hemispherical distal diaphragm with a flanged outer diameter corresponding to the inner diameter of said cap and with an opening therein for sealing the diameter of the proximal portion of said flexible tube; with
said first and second sealing washers interposed between the end flange and said end cap, whereby the flexible tube is sealed and slideable within the secondary proximal end of said base.

10. The cannula as defined in claim 6, wherein the a primary tubular proximal end of said base further includes an end fitting thereon for initially facilitating a trocar within the primary passage for inserting the primary distal tip of the base into the vessel.

11. The cannula as defined in claim 6, wherein the primary tubular proximal end of said base further includes an end fitting thereon, and having a “Y” junction attached thereto, with said “Y” junction having a first tubular branch for transferring fluids into the vessel and a second tubular branch for venting fluids from the vessel wherein the first branch and the second branch include means for selectively clamping off the respective branches of said junction.
